(19) United States
(12) Patent Application Publication
(10) Pub. No.: US 2024/0000326 A1
(43) Pub. Date: Jan. 4, 2024

(54) METHODS AND SYSTEMS FOR MEASUREMENT OF BLOOD PRESSURES

(71) Applicant: UNIVERSITY OF PITTSBURGH - OF THE COMMONWEALTH SYSTEM OF HIGHER EDUCATION, Pittsburgh, PA (US)

(72) Inventors: Aman MAHAJAN, Pittsburgh, PA (US); Ramakrishna MUKKAMALA, Sewickley, PA (US); Feng XIONG, Sewickley, PA (US); Mark A. FREITHALER, Pittsburgh, PA (US); Sanjeev Govinddas SHROFF, Pittsburgh, PA (US); Vishaal DHAMOTHARAN, Pittsburgh, PA (US); Anand CHANDRASEKHAR, Medford, MA (US)

(73) Assignee: UNIVERSITY OF PITTSBURGH - OF THE COMMONWEALTH SYSTEM OF HIGHER EDUCATION, Pittsburgh, PA (US)

(21) Appl. No.: 18/219,154

(22) Filed: Jul. 7, 2023

Related U.S. Application Data
(63) Continuation of application No. PCT/US2022/011657, filed on Jan. 7, 2022.
(60) Provisional application No. 63/135,430, filed on Jan. 8, 2021.

(51) Int. Cl. A61B 5/022; A61B 5/00; A61B 5/021; A61B 5/024

(52) U.S. Cl. CPC A61B 5/02225; A61B 5/742; A61B 5/6826; A61B 5/0077; A61B 5/02108; A61B 5/02416; A61B 2562/0219

(57) ABSTRACT

Devices and methods for determining blood pressures are provided. In one example, a device for determining blood pressure of a subject includes a camera configured to measure a finger photo-plethysmography (PPG) waveform, an accelerometer configured to measure a vertical height of the device relative to a heart of a subject, an output device configured to guide the subject to raise a hand to vary the transmural pressure of an artery while maintaining a finger pressure on the camera, and a processor configured to compute pulse pressure of the subject from the finger PPG waveform and the vertical height, and display the pulse pressure on the screen.

### CROSS-REFERENCE TO RELATED APPLICATION

This application is a continuation of International Patent Application No. PCT/US2022/011657, filed Jan. 7, 2022, which claims priority to U.S. Provisional patent application Ser. No. 63/135,430, filed Jan. 8, 2021, all of which are hereby incorporated by reference herein in their entireties.

### BACKGROUND

High arterial blood pressure (BP) afflicts many people (e.g., about one in three adults worldwide). While the incidence tends to increase with age, certain people can develop hypertension early in adulthood (e.g., about one in five US adults under 40 years old are hypertensive). The condition can be asymptomatic, but the risk for stroke and heart disease can increase monotonically with BP for a given age. Certain medications can lower BP and cardiovascular risk. However, only about three in seven people with hypertension are aware of their condition, and one of these seven has their BP under control. According to certain epidemiological data, hypertension has emerged as a leading cause of disability-adjusted life years lost.

Certain auscultatory and oscillometric BP measurement devices can be used for managing hypertension. However, these devices can lead to reduced hypertension awareness and control rates due at least in part to their reliance on an inflatable cuff. Cuff-based devices are not readily available, especially in low resource settings. As such, it can be inconvenient for people to regularly check their BP. Regular measurements during daily life are desirable, for example to circumvent white coat and masked effects in the clinic, in which patients can present with higher or lower BP than usual and to average out the large variations in BP that can occur over time, for example due to stress, physical activity, and other factors. If BP can be measured with a more convenient device, more people can be aware of their condition or be motivated to take their medications.

Therefore, there is an opportunity for methods and systems for measuring blood pressures with more convenient instrumentation.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter provides devices and methods for determining the blood pressure of a subject. A device for determining blood pressure of a subject can include a force sensor configured to measure finger pressure, a camera configured to measure a finger photo-plethysmography (PPG) waveform, a screen configured to display a visual indicator to guide a subject to place a side of a finger on the camera and the screen to target a digital artery and display the finger pressure in real time such that the subject can uniformly press the finger on the camera and screen to vary external pressure of the artery, and a processor. The processor can be configured to construct an oscillogram that can be a variable-amplitude blood volume oscillations versus external finger pressure function, compute a blood pressure of the subject from the oscillogram, and display the blood pressure on the screen. In non-limiting embodiments, the processor can be configured to determine the visual indicator based on different finger placements on the camera and screen before performing the blood pressure measurement.

The disclosed subject matter provides a device for determining blood pressure of a subject that can include a skin contact area sensor configured to measure a finger area, a camera configured to measure a finger photo-plethysmography (PPG) waveform, a screen configured to display a visual indicator to guide a subject to place a fingertip on the camera and the screen to target a transverse palmar arch artery and configured to display the finger pressure in real time to guide the subject to uniformly press the fingertip on the camera and screen to vary the external pressure of the artery, and a processor. The processor can be configured to convert the finger area to finger pressure based on a pre-defined nomogram, construct an oscillogram that can be a variable-amplitude blood volume oscillations versus external finger pressure function, compute systolic and diastolic blood pressure of the subject from the oscillogram, and display the systolic and diastolic blood pressures on the screen.

As embodied herein, the nomogram can be configured to determine finger force from the finger area based on selected parameters of a parametric function and divide the determined finger force by the finger area to determine finger pressure. For purpose of illustration and not limitation, the selected parameters can be determined based on fingertip dimensions of the subject, a single cuff blood pressure reading, or a hand raising maneuver. As embodied herein, the subject can hold the device above heart level during the finger pressing, which can provide a more accurate nomogram, and the processor can be configured to use a vertical height between the device and the heart of the subject to adjust the blood pressure measurement to the heart level.

The disclosed subject matter provides a device for determining blood pressure of a subject that includes a camera configured to measure a finger photo-plethysmography (PPG) waveform, an accelerometer configured to measure a vertical height of the device relative to a heart of a subject, an output device configured to guide the subject to raise a hand to vary the transmural pressure of an artery while maintaining a finger pressure on the camera, and a processor. The processor can be configured to compute pulse pressure of the subject from the finger PPG waveform and the vertical height and display the pulse pressure on the screen.

As embodied herein, the processor can be further configured to guide the subject to apply hard finger pressure on the camera, guide the subject to change a level of finger pressure based on the measured AC and/or DC value of the PPG waveform and the PPG measurement during the hard finger pressure, and identify a finger pressure corresponding to when a blood volume oscillation is near maximal. As embodied herein, the processor can be further configured to compare the PPG waveform during hand raising with the PPG waveform during finger pressing to assess a level of the accuracy of the device.

As embodied herein, the processor can be configured to construct a shifted oscillogram to relate variable-amplitude blood volume oscillations to a hydrostatic pressure change measured using the vertical height. The pulse pressure can be computed from the shifted oscillogram. As embodied herein, the accelerometer can be configured to measure the vertical height of the device relative to the heart. For purpose of illustration and not limitation, the processor can be configured to convert the pulse pressure to brachial artery pulse pressure using a transfer function.

The disclosed subject matter provides a device for determining blood pressure of a subject that includes a force sensor configured to measure finger pressure of the subject, a PPG sensor configured to measure a finger PPG waveform of the subject, a barometric pressure sensor configured to measure a vertical height of the device relative to a heart of the subject, and a processor. The processor can be configured to measure readings of the barometric pressure sensor during finger pressing and without the finger pressing while holding the device at heart level, adjust the blood pressure measured during the finger pressing to a heart level using the readings of the barometric pressure sensor, and display the adjusted blood pressure of the subject on a screen. As embodied herein, the blood pressure can be adjusted based on blood density, gravity, and/or the readings of the barometric sensor.

The disclosed subject matter provides a device for determining blood pressure of a subject that includes a force sensor configured to measure finger pressure and finger pressure oscillation, a visual indicator to guide a subject to place a fingertip on the force sensor, a screen configured to display the finger pressure in real time to guide the subject to press the finger on the sensor to vary the external pressure of the underlying artery, and a processor. The processor can be configured to measure AC and DC components of the finger pressure, identify an AC finger pressure pulse of maximal oscillation and a DC finger pressure at the maximal oscillation, determine a blood pressure of the subject based on the AC finger pressure pulse of maximal oscillation, and the DC finger pressure at the maximal oscillation, and display the blood pressure of the subject on the screen.

As embodied herein, the processor can be configured to determine the blood pressure based on fingertip dimensions of the subject and/or a single cuff blood pressure reading of the subject. As embodied herein, the processor can be further configured to compute diastolic blood pressure from the variable-amplitude finger pressure pulse oscillations and to compute systolic blood pressure from a blood pressure waveform

As embodied herein, the blood pressure waveform is converted to the brachial artery blood pressure waveform using a transfer function and regression equation. For purpose of illustration and not limitation, the device can further include a barometric pressure sensor to detect blood pressure at the heart level.

The disclosed subject matter provides a device for determining blood pressure of a subject that includes an array of force sensors configured to measure finger pressure and finger pressure pulse over each sensing element of the array, a visual indicator to guide the person to place a fingertip of the subject on the sensor array, a screen configured to display the finger pressure in real time to guide the subject to press the fingertip on the sensor to vary the external pressure of the underlying artery, and a processor. The processor can be configured to measure AC and DC components of the finger pressure at each sensing element of the array, determine a blood pressure of the subject from the AC and DC components, and display the blood pressure of the subject on the screen.

As embodied herein, the blood pressure can be determined based on maximal pressure pulse oscillation over the sensing elements and the DC components of the finger pressures. For purpose of illustration and not limitation, the processor can be further configured to generate a finger blood pressure waveform based on the AC and DC components and convert the blood pressure waveform to a brachial artery blood pressure waveform using a transfer function and regression model. As embodied herein, the device can further include a barometric pressure sensor to detect the blood pressure at the heart level.

The disclosed subject matter provides a device for determining blood pressure of a subject that includes a force sensor configured to measure finger pressure and a finger pressure pulse, a finger photo-plethysmography (PPG) sensor configured to measure a PPG waveform, a visual indicator to guide a subject in placing a fingertip on the sensors, a screen configured to display the finger pressure in real time to guide the subject to press the fingertip on the sensors to vary the external pressure of the underlying artery, and a processor. The processor can be configured to measure AC and DC components of the finger pressure and the PPG waveform, compute an arterial compliance curve using the AC finger pressure component and PPG waveform, compute a blood pressure of the subject using the arterial compliance curve, and display the blood pressure of the subject on the screen.

As embodied herein, the processor can be further configured to compute the blood pressure by forming an oscillogram based on external finger pressure and PPG waveform, performing a cross-correlation between the arterial compliance curve and the derivative of the oscillogram with respect to pressure, and determining a minimum value and a maximum value of the cross-correlation as systolic and diastolic blood pressures.

### BRIEF DESCRIPTION OF THE DRAWINGS

FIG. 1 is a diagram and graphs illustrating an example oscillometric finger pressing technique for cuff-less and calibration-free monitoring of arterial blood pressure (BP) via a mobile device in accordance with the disclosed subject matter.

FIGS. 2A-2B are diagrams illustrating an example smartphone-based device with a custom photo-plethysmography (PPG)-force sensor unit to implement the oscillometric finger pressure techniques and comparisons of the cuff-less device to cuff devices in accordance with the disclosed subject matter.

FIGS. 3A-3C are photographs illustrating an example mobile device application to implement the oscillometric finger pressing technique via PPG and force sensors in the smartphone in accordance with the disclosed subject matter.

FIG. 4 is a photograph illustrating an example of techniques for BP measurement from the digital artery using PPG and 3D Touch sensors in smartphones in accordance with the disclosed subject matter.

FIG. 5 is a graph illustrating an example volume clamping technique for measuring the BP waveform via a finger cuff-PPG device in accordance with the disclosed subject matter.

FIG. 6 is a diagram illustrating an example technique for measuring pulse pressure using a standard smartphone without 3D Touch capabilities in accordance with the disclosed subject matter.

FIG. 7 is a chart illustrating an example method of computing finger force from finger screen contact area measurements to measure systolic and diastolic BP via the oscillometric finger pressing method and a standard smartphone in accordance with the disclosed subject matter.

FIG. 8 is a chart illustrating finger pressure measurement versus time during finger pressing in accordance with the disclosed subject matter.

FIG. 9 is a diagram illustrating an example finger pressing technique with a pressure sensor alone based on an applanation tonometry technique in accordance with the disclosed subject matter.

FIG. 10 is a diagram illustrating an example method of leveraging the AC components of both the PPG and pressure measurements in conjunction with a physiologic model to compute BP in accordance with the disclosed subject matter.

FIG. 11 is a diagram illustrating an example method for computing venous blood pressure (VP) from the DC component of the finger PPG waveform during the variation in finger transmural pressure in accordance with the disclosed subject matter.

FIG. 12 is a diagram illustrating an example method of measuring VP via a volume clamping finger cuff-PPG device by varying the setpoint and detecting VP via the counter cuff pressure measurement in accordance with the disclosed subject matter.

### DETAILED DESCRIPTION

Reference will now be made in detail to the various exemplary embodiments of the disclosed subject matter, which are illustrated in the accompanying drawings.

The terms used in this specification generally have their ordinary meanings in the art, within the context of the disclosed subject matter, and in the specific context where each term is used. Certain terms are discussed below, or elsewhere in the specification, to provide additional guidance to the practitioner in describing the compositions and methods of the disclosed subject matter.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing,” and “comprising” are interchangeable, and one of skill in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

A “user” or “subject” herein is a vertebrate, such as a human or non-human animal, for example, a mammal. Mammals include, but are not limited to, humans, primates, farm animals, sport animals, rodents, and pets.

The disclosed subject matter provides techniques for determining the blood pressure of a subject. The disclosed subject matter provides systems and methods for determining blood pressure of a subject using a non-invasive cuff-less device. A non-invasive cuff-less device as embodied herein can be configured as or utilize a portable device, which can be configured as a stand-alone medical device using specialized hardware and/or software as described herein. Additionally or alternatively, a non-invasive cuff-less device can utilize a general-purpose mobile or wearable device, such as a smartphone, portable computer, or other suitable general-purpose device. An example non-invasive cuff-less device 100 can include a camera 304, a sensor 101, a screen 102, and a processor.

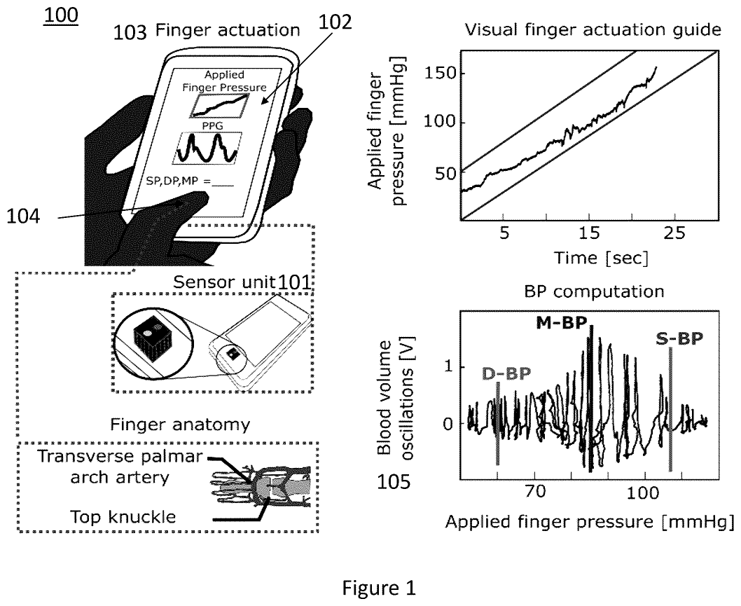

As embodied herein, and as shown for example in FIG. 1, the screen 102 can be used for displaying a visual indicator to guide the subject to place a finger (e.g., side of a finger) on the camera and the screen (e.g., to target measurement from a digital artery and display the finger pressure in real time such that the subject can uniformly press the finger on the camera and screen to vary the external pressure of the artery). Additionally or alternatively, other output devices can be used to guide the subject as described herein. Such output devices can include visual output devices, such as screen 102 or other visual device configured to provide a visual indicator, animation, text or other visual signals to guide the subject as described herein. In addition or as a further alternative, the output device can include a speaker or other audio device configured to provide an audio indicator, spoken text or instructions, or other audio signals to guide the subject as described herein.

For example, and as embodied herein, the device 100 can provide a one-time or periodic initialization to determine optimal finger placement for the user. In this initialization, a user is guided to incrementally place more of the finger on the screen. The device 100 can identify the finger positioning that provides a suitable area of screen contact without approaching force saturation. For example and without limitation, device 100 can provide a visual indicator that can guide the user to place the finger that can yield a largest area of screen contact without force saturation.

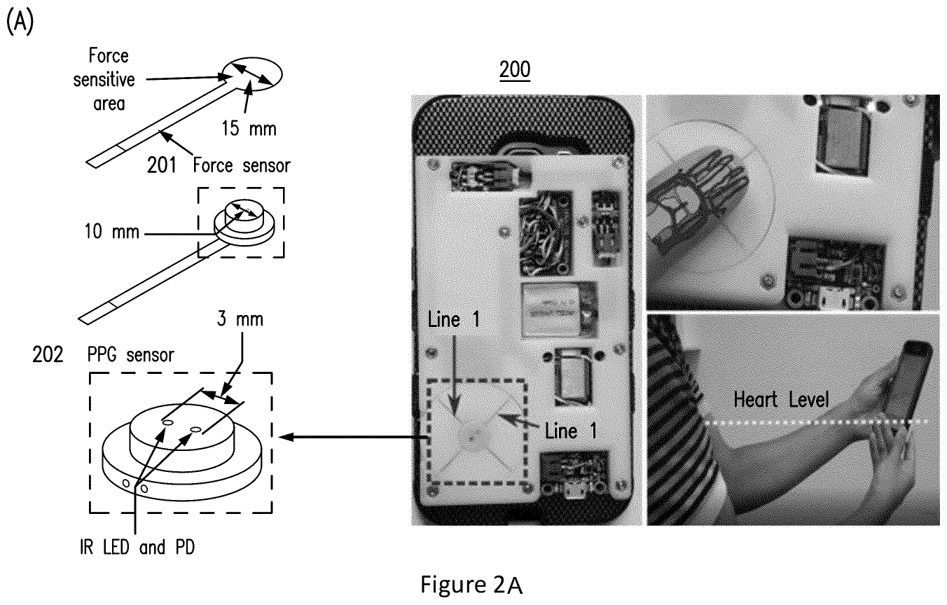

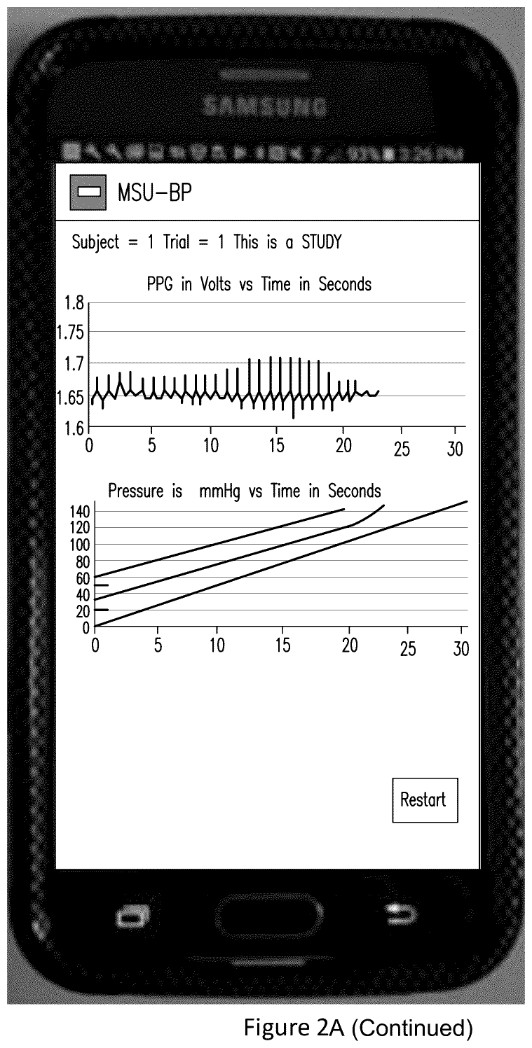

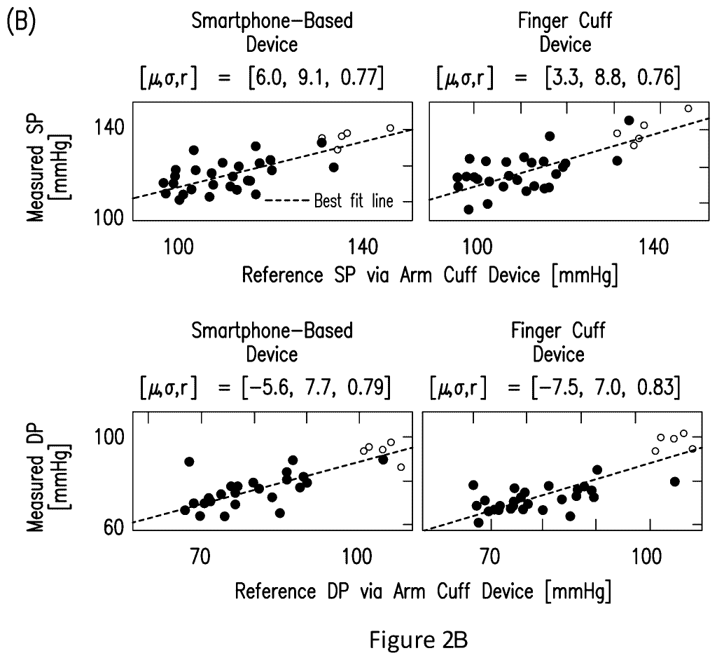

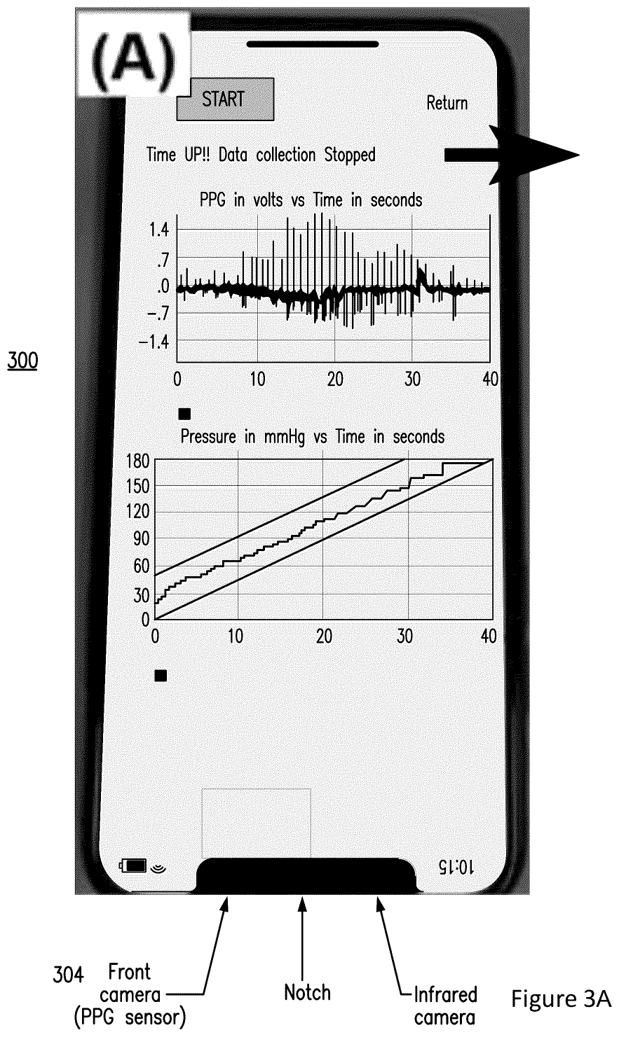

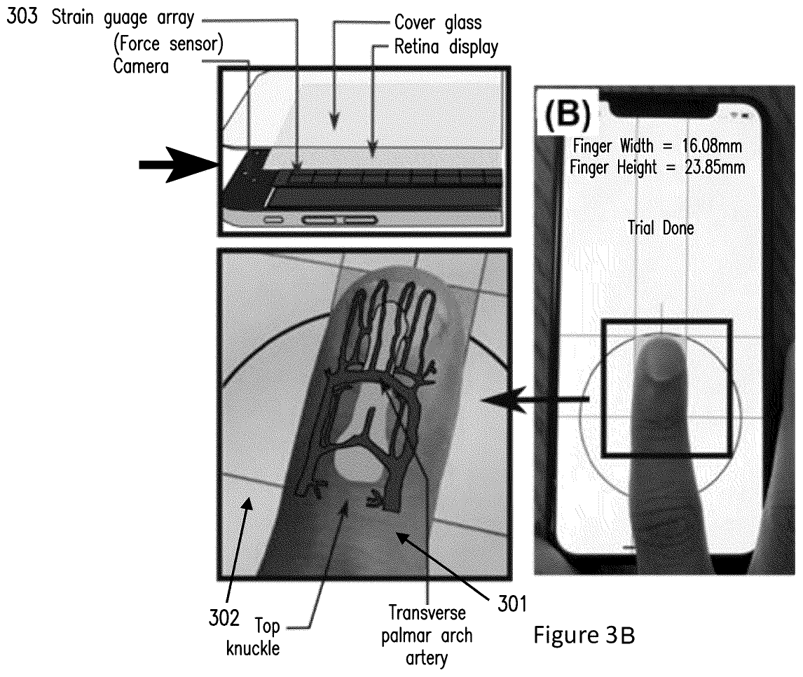

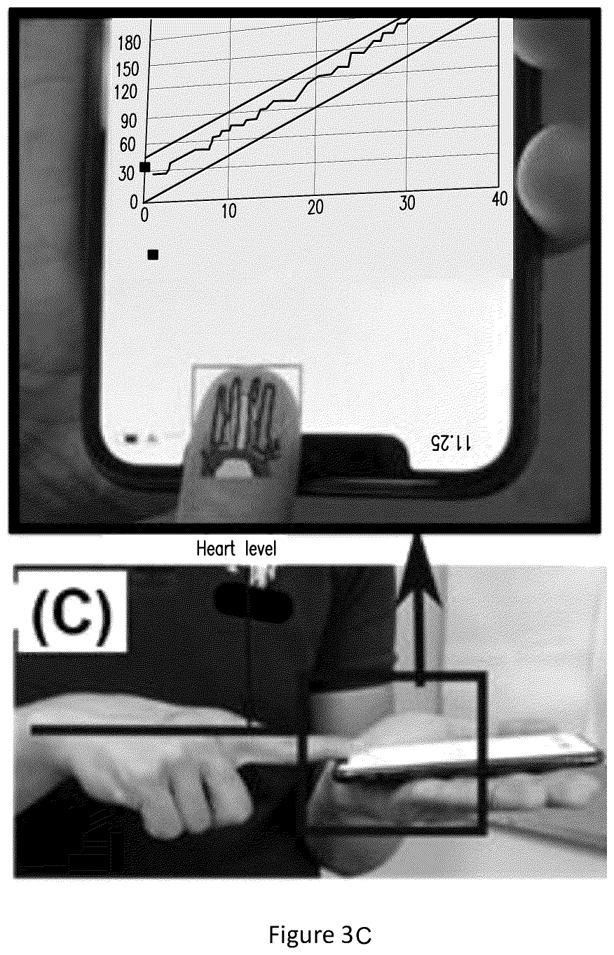

As embodied herein, the disclosed device 200 can include a force sensor 101. The force sensor can measure the finger pressure on the force sensor. For example and without limitation, with reference to FIG. 2A, the force sensor 201 can be coupled to a plethysmography (PPG) sensor 202 for measuring both finger pressure and PPG waveforms of the subject. As embodied herein, the force sensor can be located under the screen (e.g., 3D touch sensor). For example, as shown in FIG. 3B, a user can press their finger 301 onto the screen 302, and the force sensor 303 under the screen 302 can measure the applied force for determining blood pressure. The screen can be configured to display the finger pressure in real time to guide the subject to uniformly press on the sensors to vary the external pressure of the artery.

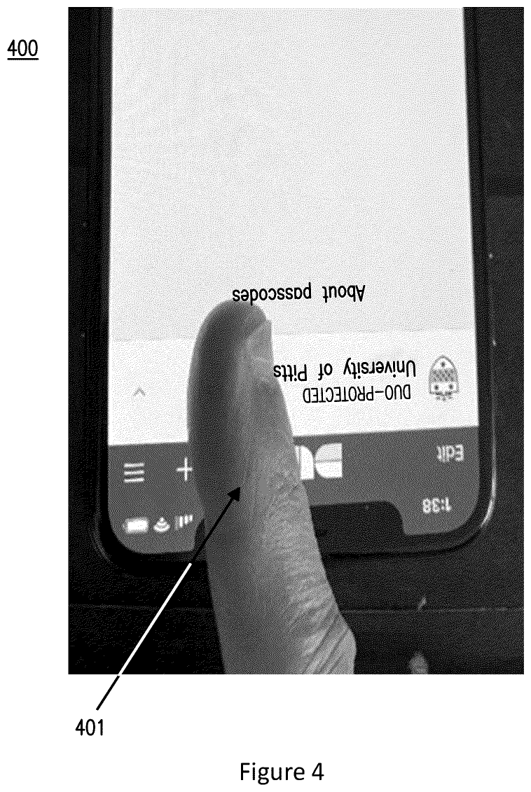

Referring still to FIG. 3A, as embodied herein, the camera 304 can be configured to measure finger photo-plethysmography (PPG) waveforms. For example, a user can uniformly press their finger onto the camera and screen (e.g., starting at 30 mmHg and approaching 180 mmHg), and the camera can measure the PPG waveforms of the user. With reference to FIG. 4, for example and without limitation, the side of the finger 401 can be pressed against the camera to obtain the PPG waveforms. As embodied herein, other components of the device 400 can be simultaneously used for improving the accuracy of the device 400. For example, by uniformly pressing the side of the finger against the front camera and screen, both PPG and force measurements can be obtained with improved accuracy.

As embodied herein, the device 100 can include a processor. The processor can be configured to form an oscillogram based on the measured PPG waveform and finger pressure. For example, the oscillogram can be a variable-amplitude blood volume oscillations versus an external finger pressure function 105. The variable-amplitude blood volume oscillations can be obtained from the PPG waveform as the user presses their finger on the sensors to vary the external pressure of the underlying artery. The processor can determine the blood pressure from the oscillogram and display the determined blood pressure on the screen. For example, the processor can estimate systolic and diastolic blood pressure from the oscillogram using the standard fixed ratio algorithm, a patient-specific algorithm, or another suitable oscillometric BP estimation algorithm. Additionally or alternatively, mean blood pressure can be estimated using similar techniques.

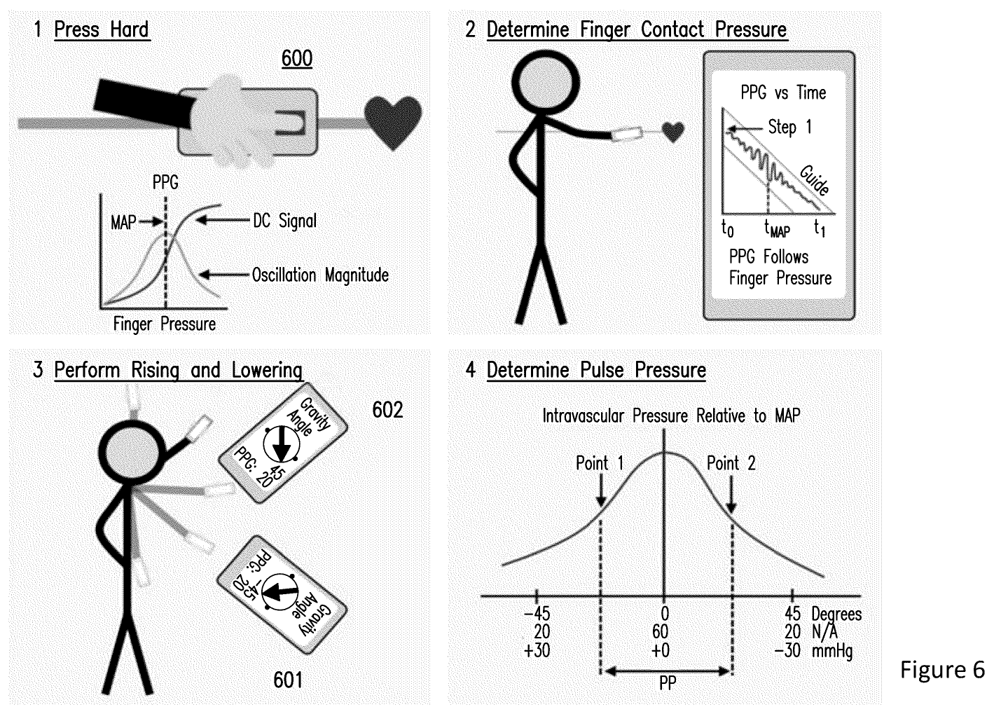

The processor can alternatively estimate the pulse pressure (e.g., PP=systolic blood pressure minus diastolic blood pressure) based on the PPG waveform that can be obtained through a PPG sensor (e.g., camera or finger PPG sensor) without using a force sensor. The finger PPG waveform can include alternating current (AC) and direct current (DC) components during increasing external finger pressure. For example, the processor can use the DC and/or AC components of the PPG waveform to determine how much finger pressure the user needs to apply on the PPG sensor (e.g., camera or finger PPG sensor). As shown for example in FIG. 6, the user first can press hard on the PPG sensor to determine a highest DC value based on the user input. The device 100 can show a graph for recording the DC value versus time, where the y-axis range can set by the identified highest DC level. The processor can determine the DC level at which the AC oscillation amplitude is greatest, which can correspond to mean BP and show a constant target line to guide the user in attaining this level of substantially contact pressure. For purpose of illustration and not limitation, as shown in FIG. 6, while maintaining this substantially constant finger pressure, the user can lower the device 600 pointing downwards 601 to the floor and slowly raise the device 600 above their head with the device 600 pointed upwards 602 (or vice versa). The hand-raising actuation can be performed in a continuous motion (e.g., over 20-40 s) or incrementally in steps (e.g., approximately 30 degrees for a 3-5 s at a time guided by the smartphone via audio cues). The device 600 can include an accelerometer/gyroscope to measure the internal hydrostatic blood pressure change. The processor can generate a variable amplitude blood volume oscillation versus hydrostatic blood pressure change function based on the obtained values. As embodied herein, the function can be a shifted oscillogram. The PP can be computed from the width of the oscillogram using a fixed ratio algorithm or another similar algorithm. For purpose of illustration and not limitation, the shifted oscillogram can be constructed to relate the variable-amplitude blood volume oscillations to the hydrostatic pressure change measured using the vertical height. For example, a user can be instructed to hold the device 600 in a pre-determined position and orientation such that one axis of the accelerometer can be used to determine the vertical height relative to the heart. For example, and as embodied herein, the top of the phone is pointed upwards when the phone is fully raised above the head and pointed downwards when the phone is fully lowered. In this case, the accelerometer in the top-to-bottom direction of the phone can be used to determine the vertical height relative to the heart. Alternatively, the hydrostatic BP change can be estimated without using an accelerometer/gyroscope or any other sensor. While maintaining the substantially constant finger pressure, the user can lower the device 600 pointing downwards 601 to the floor and raise the device 600 above their head in intuitive and fixed increments (e.g., approximately 45 degrees for a 3-5 s at a time guided by the smartphone via audio cues). The hydrostatic BP change can then be estimated based on the known increments. The advantage here is that the device 600 need not be held in any pre-determined position, so maintaining the constant finger pressure during the hand raise may be easier to do. As embodied herein, the disclosed device 100 can perform a quality assessment. For example, the processor can assess the quality of blood pressure measurements by comparing the PPG waveform during hand raising with the PPG waveform during finger pressing.

As embodied herein, the processor can convert the finger BP measurement into brachial artery BP. For example, to obtain brachial blood pressure, the processor can extract the PPG waveform beat with maximal oscillation. The PPG waveform beat can be then calibrated so that its minimum and maximum can correspond to the computed finger diastolic and systolic BP. A transfer function to account for a wave reflection and a regression model to account for the resistive pressure drop, can be applied to convert the finger waveform beat to a brachial BP waveform beat. The minimum and maximum of the brachial BP waveform beat can be defined as systolic and diastolic blood pressure. Finger PP can be converted to brachial artery PP in a similar way but without using the regression model.

As embodied herein, the device 100 can include a skin contact area sensor, and the processor can be configured to measure systolic and diastolic blood pressures using the skin contact area sensor. The processor can convert the measured finger area to finger pressure using a pre-determined nomogram. The nomogram can include a parametric function (e.g., an exponential) to predict the finger force/pressure from the finger area. The processor can determine the parameters of this function by using an empirical equation with fingertip dimensions as input and the parameter(s) as the output and/or utilizing two-finger pressure measurements at two different heights relative to the heart for a known blood pressure change or a single cuff blood pressure measurement. The empirical equation can be derived from a training dataset from a cohort of subjects. Finger pressure can be obtained by computing force from the area measurement via the parametric function and dividing this value by the measured area. For purpose of illustration and not limitation, the user can hold the device 100 above the heart level to improve the accuracy of the device 100. For example, the user can lie down and hold the device 100 upward with arms straight while performing the finger pressing actuation. As embodied herein, the computed blood pressure can be adjusted for the hydrostatic blood pressure change using the arm length.

As embodied herein, the device 100 can include a barometric pressure sensor for detecting BP at heart level. For example, the barometric pressure sensor can detect height differences of less than about 5 cm, which can correspond to a minor error of blood pressure (e.g., less than about 3.5 mmHg error). A user can hold the device 100 with the barometric pressure sensor at heart level in a pledge of allegiance pose for about 5-20 sec. The barometric pressure measurement can be averaged. The user can perform the finger pressing technique as described herein to measure blood pressure while holding the smartphone in a static, arbitrary way including any vertical level relative to the heart. For purpose of illustration and not limitation, the barometric pressure can be averaged over the finger actuation. The difference in the two measurements can provide the vertical height for correcting the blood pressure measurement for the hydrostatic BP difference. As embodied herein, the blood pressure measurement can be corrected to heart level based on the blood density, gravity, and barometric sensor readings. For example, values of rho-g-h, where rho is the known blood density, g is gravity, and h is the second barometric sensor reading minus the first barometric sensor reading, can be added to the blood pressure measurement to correct it to heart level. For purpose of illustration and not limitation, the disclosed device 100 can include a temperature sensor that can be used to assess the quality of PPG and barometric measurements.

As embodied herein, the barometric pressure sensor can be used alternatively or in addition to the accelerometer to determine the vertical height of the disclosed device 100 relative to the heart during the hand raising technique 601, 602.

As embodied herein, the disclosed device 100 can determine the blood pressure of a user/subject with a pressure sensor alone. For example, the user can press on a force sensor of the device 100 on an artery. For purpose of illustration and not limitation, the disclosed device 100 can include the force sensor of a known area. For example, a user can perform the finger pressing as described herein, and congruent with the conventional applanation tonometry principle, the AC pressure waveform can increase with reducing wall tension and then decrease with arterial occlusion. The AC waveform beat at maximal amplitude can correspond to a zero-mean blood pressure waveform beat (ΔP(t)) scaled by the unknown constant that could be related to the area of the artery divided by the area of the sensor (k). Mean blood pressure (P_m) can be given by the DC pressure at which the AC amplitude is maximal according to applanation tonometry. The processor can generate a waveform indicating kΔP(t)+P_m based on the measurements. The parameter k can be determined to have a fully defined finger blood pressure waveform beat. For purpose of illustration and not limitation, the parameter k can be determined via an empirical equation relating arterial area to fingertip dimensions or by calibration with a single cuff blood pressure measurement. As embodied herein, the parameter k can be determined based on the measured diastolic blood pressure (P_d) from the AC pressure waveform. The minimum of ΔP(t) can be scaled to equal P_d-P_m, and P_m can be added to the waveform to generate the finger BP waveform. The peak of the waveform can be systolic blood pressure (P_s).

As embodied herein, the disclosed device 200 can measure finger blood pressure using tonometric finger pressing. According to the applanation tonometry principle, a force sensor can flatten or applanate the artery so that the wall tension can be perpendicular to the force sensor and can be encompassed by the flattened artery so that pressure can be derived as the ratio of the measured force to the known sensor area. For example, the disclosed device 200 can include a multi-sensor force array. The multi-sensor array can be attached to the back of the device 200 (e.g., a mobile device or smartphone). A user can perform the finger pressing actuation, and the maximum force oscillation beat over finger pressures, and all sensors can be detected. This beat can be divided by the sensing element area to yield a finger BP waveform beat. For purpose of illustration and not limitation, the sensor array can include sensing elements that can be smaller than the entire sensor. The smaller sensing elements can thus be suitable for use in the sensor array without as high resolution specifications.

As embodied herein, the disclosed device 100 can utilize both a sensitive force sensor and the PPG sensor for the improved accuracy of blood pressure computation. The PPG sensor can be the camera or the finger PPG sensor. The processor can compute systolic and diastolic blood pressures from the AC components of both PPG and pressure measurements. For example, the processor can use an oscillometry model to compute the blood pressure based on the AC components of the PPG and pressure measurements. The device 100 can include a PPG-force sensor unit that can detect the AC pressure waveform. The user can perform the finger pressing method with the sensor unit as described herein. The AC pressure waveform beat of maximal amplitude, which can be concatenated to correspond with the multiple PPG waveform beats, can be selected. The derivative of the PPG waveform can be selected with respect to the concatenated pressure waveform and plotted against the DC pressure measurement to yield data points indicating a shifted arterial compliance curve. For example, a parametric function can be fitted to the data points, and the function can be shifted so that its peak is at zero transmural pressure to arrive at the scaled arterial compliance curve. For purpose of illustration and not limitation, the blood pressure can be computed by forming the oscillogram, taking its derivative with respect to external finger pressure, and performing a cross-correlation between the arterial compliance curve and derivative of the oscillogram. The peak location of the cross-correlation function can denote diastolic BP, and the valley location can denote systolic blood pressure. Alternatively, an oscillometry model with the compliance curve can be fitted to the measured oscillogram or its derivative in an optimal sense to estimate blood pressure.

### EXAMPLES

The following examples are provided for purpose of illustration and confirmation of the disclosed subject matter only and without limitation thereof.

### BACKGROUND

High arterial blood pressure (BP) afflicts about one in three adults worldwide. While the incidence increases with age, many people develop hypertension early in adulthood (e.g., more than one in five US adults under 40 years old are hypertensive). The condition is usually asymptomatic, but the risk for stroke and heart disease increases monotonically with BP for a given age. Lifestyle changes and many inexpensive, once-daily medications can lower BP and cardiovascular risk. Yet, only about three in seven people with hypertension are aware of their condition, and just one of these seven has their BP under control. Epidemiological data on hypertension in low resource settings are more alarming. As a result, hypertension has emerged as the leading cause of disability-adjusted life years lost.

Auscultatory and oscillometric BP measurement devices have been instrumental in managing hypertension. At the same time, these devices may bear responsibility for the abysmal hypertension awareness and control rates due to their reliance on an inflatable cuff. Cuff-based devices are not readily available, especially in low resource settings. Hence, most people do not regularly check their BP. Regular measurements during daily life are needed to circumvent white coat and masked effects in the clinic in which patients present with higher or lower BP than usual and to average out the large variations in BP that occur over time due to stress, physical activity, and other factors. If BP could be measured more conveniently, then many people would become aware of their condition or motivated to take their medications.

Hence, cuff-less BP monitoring devices are being widely pursued. However, the devices under investigation generally suffer from the debilitating limitation of requiring calibrations with cuff devices in order to output a measurement in units of mmHg.

The oscillometric principle has been extended for cuff-less and calibration-free BP monitoring via readily available smartphones. FIG. 1 illustrates the concept. The user serves as the actuator (instead of a cuff) by pressing their fingertip against the phone (held at heart level) to steadily increase the external pressure of the underlying artery. Meanwhile, the phone, embedded with basic photo-plethysmography (PPG) and force transducers, serves as the sensor (rather than the cuff device) to measure the resulting variable-amplitude blood volume oscillations and applied finger pressure. The phone also provides visual feedback to guide the finger actuation and applies an algorithm to compute BP from the measurements just like a cuff device. A video demonstration is available.

A device was developed comprised of a custom PPG-force sensor unit affixed to the back of a smartphone to implement this “oscillometric finger pressing method”. The device could yield BP measurements with a level of accuracy comparable to an FDA-cleared finger cuff volume clamping device over the normotensive range. FIGS. 2A-2C illustrate the device and accuracy results. The oscillometric finger pressing method could be implemented simply as an iPhone X application by leveraging the front camera as the PPG sensor and the sensitive strain gauge array (“3D Touch”) under the screen as the force sensor. FIGS. 3A-3C illustrates the app.

Techniques for measurement of blood pressures have included using a mobile device with PPG and force sensors and visual indicia to indicate where to place the fingertip on the sensor unit, steadily varying fingertip pressure under guidance of the smartphone, forming an oscillogram (variable-amplitude blood volume oscillations versus applied pressure function), and computing BP from the oscillogram. These techniques provided a convenient site for BP measurement via finger pressing—the transverse palmar arch artery in the fingertip (see FIG. 1).

The disclosed subject matter includes related improvements to further advance measurement of blood pressures. These improvements generally circumvent prior techniques.

Improved Apps for Smartphones with Existing PPG and Force Sensors

There are many smartphones with 3D Touch capabilities including iPhone 6s-X models and select Huawei and Xiaomi models. In these phones, the front camera for PPG sensing is at some distance (e.g., 2-8 mm) from the force sensor under the screen, and the base of the nail on the fingertip should be above the front camera for high-fidelity measurement of the blood volume oscillations. Hence, only a small portion of the finger will be on the force sensor under the screen, which will degrade the measurement of force while rendering small changes in finger force to translate to large changes in pressure (i.e., more difficult finger actuation). Conversely, placing a greater portion of the fingertip on the screen will degrade the PPG measurement.

The disclosed subject matter provides techniques configured to target a digital artery running along a side of the finger (see FIGS. 1, 2A, 2B, 3A, 3B. and 3C) to measure BP via PPG and 3D Touch sensors already in the smartphone. FIG. 4 illustrates the finger positioning concept. By uniformly pressing the side of the finger against the front camera and screen, both PPG and force measurements may be obtained with superior accuracy. However, placing too much of the finger on the screen can saturate the force measurement at lower pressures. A solution is to employ a one-time or periodic initialization (see, e.g., FIG. 3B) to determine optimal finger placement for a given user. In this initialization, the user incrementally places more of the finger on the screen and performs the finger pressing method for each finger placement. The data are analyzed to determine the finger positioning that yields the largest area of screen contact without approaching force saturation. Alternatively, or in addition, the phone could be held above the heart (e.g. at shoulder level), which would decrease the BP in the finger and thus the possibility of early saturation of the force sensor. Then, the known or measured distance between heart and shoulder levels could be used to correct the computed BP for this “hydrostatic BP change” (see next section for details).

Apps for Conventional Smartphones without Force Sensors

Given that virtually every adult has real risk for developing hypertension and that smartphones are available to billions of people including those in low resource settings, it is desirable to make standalone smartphones into BP monitors. However, most smartphones do not include 3D Touch or similarly sensitive force sensors. As such, the disclosed subject matter provides techniques to measure absolute BP in units of mmHg using only standard smartphones.

One example uses arm rather than finger actuation. In oscillometry, the cuff compresses the artery to vary its external pressure. During this process, the device also measures the cuff pressure, which indicates both the blood volume oscillations in the artery (AC cuff pressure) and the external pressure (DC cuff pressure). BP is estimated from the resulting oscillogram, which is again the function relating the variable-amplitude blood volume oscillations to the applied pressure. Note that the abscissa of the oscillogram may be viewed more generally as a change in transmural pressure of the artery (i.e., internal BP minus external cuff pressure in this case). Certain methods thus involve varying the internal rather than external pressure of an artery to change the transmural pressure. As a user of a finger worn ring device lowers their hand with arm straight, the internal BP in the finger increases due to the weight of the arm blood column (“hydrostatic effect”) by an amount equal to pgh, where p is the known density of blood, g is gravity, and h is the vertical distance between the hand position and heart. In this way, arterial transmural pressure is varied without a cuff. The device includes a PPG sensor, force sensor, and accelerometer. The accelerometer allows measurement of the hydrostatic BP change (i.e., plg sin θ, where l is the measured arm length, θ is the angle between the arm and horizontal plane, and g sin θ is the accelerometer output). The BP changes for typical arm lengths is about ±50 mmHg with respect to heart level. For a mean BP of 80 mmHg, the transmural pressure variation is about 30 to 130 mmHg. However, the oscillogram in both the positive and negative transmural pressure regimes is needed to compute BP accurately. The ring must thus be worn tight enough to generate negative transmural pressures. The force sensor of known area measures the ring contact pressure on the finger, which is subtracted from the hydrostatic BP change. BP may then be estimated from the PPG oscillations as a function of the transmural pressure change. The main problem is that the ring should be applied with a pressure equal to around mean BP, but BP is what is sought for measurement.

Another problem with bringing the hand raising actuation to a smartphone is eliminating the need for the force sensor. However, note that all smartphones have PPG sensors in the form of a camera or a dedicated sensor (e.g., Samsung Galaxy S series) and three-axis accelerometer/gyroscope combination.

To solve these and other problems, the disclosed subject matter can limit the measurement to pulse pressure (PP=systolic BP−diastolic BP). PP would be useful to detect isolated systolic hypertension, which is a common form of hypertension that occurs with aging.

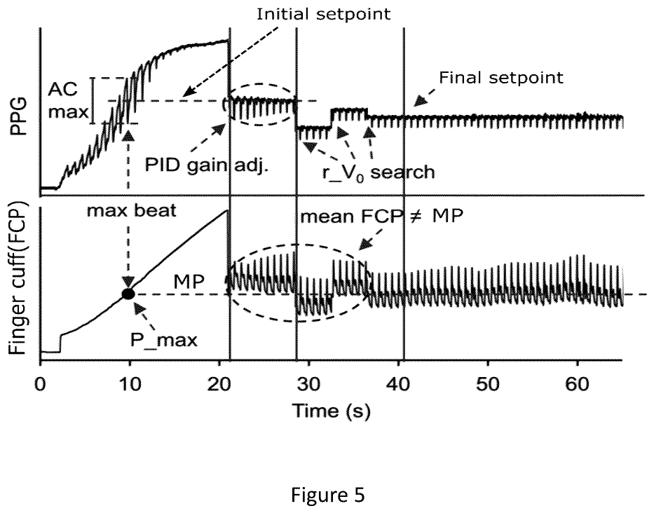

To explain the overall idea, FIG. 5 (left) shows the finger PPG waveform (AC and DC components) during increasing external finger cuff pressure. (Note that the PPG waveform is not inverted, which is typically done in practice.) The AC component increases and then decreases in amplitude, which is consistent with the oscillometric principle, while the DC components rises to a maximal value. This maximal value changes over time for a given user.

FIG. 6 shows exemplary steps to execute the overall idea of measuring absolute PP with only a standard smartphone. The first step is to use the DC and/or AC components of the PPG waveform to determine how much finger contact pressure the user should apply on the smartphone PPG sensor. For example, the user first presses very hard on the PPG sensor to determine the maximum DC value. Then, the smartphone shows a graph for recording the DC value versus time, wherein the y-axis range is set by the maximum DC level. The graph also includes two target lines, indicating that the user should press increasingly harder to reach the maximum DC level in, for example, 20-40 sec. (The user could also slowly release the finger pressure from hard pressing, as shown in the figure.) The current DC level (e.g., average of a few beats) is shown in real time to guide the finger actuation. The smartphone then finds the DC level at which the AC oscillation amplitude is near maximal, which corresponds to mean BP, and shows a constant target line to guide the user in attaining this level of contact pressure. While maintaining this constant finger pressure, the user lowers the phone pointing downwards to the floor and slowly raises it above their head with the phone now pointed upwards (or vice versa). The hand raising actuation could be performed in a continuous motion (e.g., over 20-40 s) or incrementally in steps (e.g., approximately 30 degrees for a 3-5 s at a time guided by the phone via audio cues). The y-axis accelerometer/gyroscope is used to measures the internal hydrostatic BP change. The resulting variable amplitude blood volume oscillation versus hydrostatic BP change function is a horizontally shifted oscillogram. The horizontal shift is unknown, as the finger contact pressure is not measured. For this reason, systolic and diastolic BP cannot be computed. However, PP can be computed from the width of the oscillogram using the standard fixed-ratio algorithm or otherwise. To check that the user has maintained the contact pressure throughout the hand raising actuation, the maximum oscillation can be compared to the initial maximum oscillation. If the two values differ substantially, the user is asked to try again.

Alternatively, the hydrostatic BP change can be estimated without using an accelerometer/gyroscope or any other sensor. While maintaining the constant finger pressure, the user lowers the phone to the floor and raises it upwards in intuitive and fixed increments (e.g., approximately 45 degrees for a 3-5 s at a time guided by the smartphone via audio cues). The hydrostatic BP change can then be estimated based on the known increments. The advantage here is that phone orientation, which can affect accelerometer/gyroscope usage, becomes unimportant such that the hand raising may be easier to perform. In addition, the initial step of determining the constant finger pressure may not be necessary. The user may simply press firmly on the PPG sensor and perform the hand raising. If an inverted U-shaped oscillogram is not observed, then the phone can ask the user to try again or perform the initial step. As another alternative, a smartwatch (e.g., an Apple Watch) with a PPG sensor to measure the PPG waveform from the back of the wrist could be used instead. The initial step can be performed by tightening the watch. The same watch tightness can be used for subsequent BP measurement.

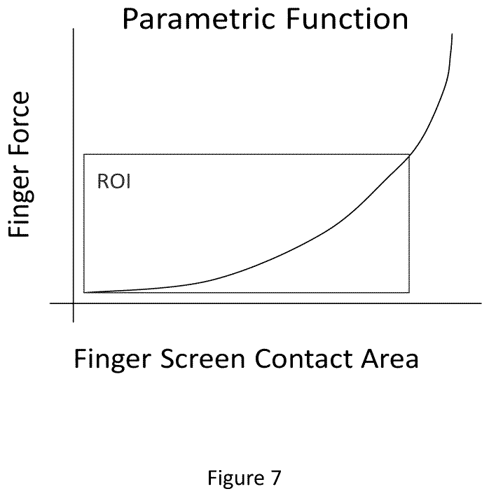

Another example includes techniques to measure systolic and diastolic BP via a standard smartphone by exploiting the existing capacitive sensor array under the screen for accurately measuring finger contact area in addition to the front camera. As shown in FIG. 7, a parametric function such as an exponential can relate finger area to force. The 1-3 unknown parameters of this function can be determined for a given user in various ways. One way is to form an empirical equation with fingertip dimensions measured with the phone (see, e.g., FIG. 3B) as input and the parameter(s) as the output. Alternatively, or in addition, two finger pressing measurements at two different heights relative to the heart for a known BP change and/or a single cuff BP measurement could be obtained, and the parameters could be determined such that the finger pressing method yields the BP values. Then, finger pressure can be obtained by computing force from the area measurement via the parametric function and dividing this value by the measured area. The function becomes less accurate at higher pressures, wherein small changes in area translate to large changes in pressure (see FIG. 7). Therefore, the lower pressure range should be preferentially used (i.e., “ROI” or region of interest in FIG. 7). One way to ensure a lower pressure range is for the user to hold the phone above heart level to decrease the blood pressure. For example, the user could lie down and hold the phone upward with arms straight while performing the finger pressing actuation. The computed BP via the finger pressing method can then be adjusted for the hydrostatic BP change using the arm length.

For either example, it is the finger BP that is measured. But brachial BP can be clinically important. Finger BP is lower by about 10 mmHg than brachial BP due to a resistive pressure drop. Finger PP is higher than brachial PP due to wave reflection, especially in more compliant arteries. Hence, finger diastolic and mean BP are lower than brachial diastolic and mean BP, while finger systolic BP is variable relative to brachial systolic BP. To obtain brachial BP, the PPG waveform beat with maximal oscillation is extracted. This beat may best but imperfectly correspond to a finger BP waveform beat. The PPG waveform beat is then calibrated so that its minimum and maximum correspond to the computed finger diastolic and systolic BP. A transfer function (to account for wave reflection) and a regression equation (to account for the resistive pressure drop) or other similar transformations are then applied to convert the finger waveform beat to a brachial BP waveform beat. The minimum and maximum of the brachial BP waveform beat are taken as systolic and diastolic BP. If only finger PP is available, then the PPG waveform beat of maximal amplitude is calibrated so that its amplitude equals finger PP, and a transfer function is then applied to obtain a zero-mean brachial BP waveform. The peak-to-peak amplitude of this waveform gives brachial PP.

Convenient Sensor for BP Measurement at Heart Level

Prior techniques included steps towards ensuring BP measurement at heart level via processing of images of the user. Such image processing is difficult and may not be accurate enough. The disclosed subject matter provides techniques to use sensitive barometric pressure sensors having increased sensitivity for hydrostatic BP correction. These sensors are able to detect height differences of <5 cm, which corresponds to only about <3.5 mmHg error. Exemplary steps are as follows. A device like the one in FIGS. 2A and 2B is developed but includes the barometric pressure sensor as well. First, the user holds the phone at heart level in a “pledge of allegiance” pose for 5-20 sec. The barometric pressure measurement is averaged. Then, the user performs the finger pressing method to measure BP while holding the phone in a static, arbitrary way including any vertical level relative to the heart. The barometric pressure is averaged over the finger actuation. The difference in the two measurements gives the vertical height for correcting the BP measurement for the hydrostatic BP difference. The barometric pressure sensor may also be accompanied by a temperature sensor that can be used to judge the quality of PPG measurement, which degrades with cold fingers and other factors.

Custom Device with Pressure Sensor Alone

PPG sensors, especially those employing visible light, do not work well in low signal conditions, such as in cold environments (e.g., air-conditioned rooms) and dark skin. Using only a pressure sensor that can measure the pulse and pressure over the BP range (e.g., 0-250 mmHg) could overcome the limitations of PPG while simplifying the sensor design or even providing greater accuracy in BP measurement. The disclosed subject matter can include implementing the finger pressing method with a pressure sensor alone based on the applanation tonometry principle. The general principle involves pressing a force sensor on an artery. In this example, the sensor must (i) flatten or “applanate” the artery so that the wall tension is perpendicular to the sensor and (ii) be encompassed by the flattened artery so that pressure may be derived as the ratio of the measured force to the known sensor area.

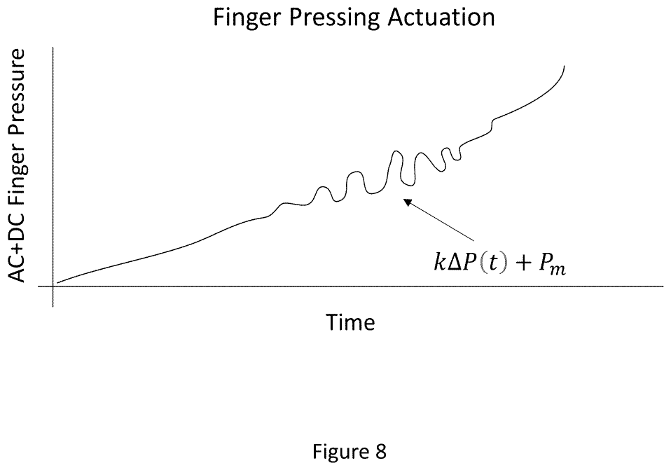

A device similar to FIG. 2A is developed, but only including a sensitive force sensor of known area. A user performs the finger pressing procedure. FIG. 8 illustrates the resulting finger pressure as a function of time during the finger pressing actuation. The AC pressure waveform increases with reducing wall tension and then decreases with arterial occlusion. The pattern is thus similar to oscillometry. The AC waveform beat at maximal amplitude may correspond to a zero-mean BP waveform beat (ΔP(t)) scaled by a constant that can be related to the unknown area of the artery divided by the area of the sensor (k). Mean BP (Pₘ) may be given by the DC pressure at which the AC amplitude is maximal. Hence, this process yields a waveform indicating kΔP(t)+Pₘ. The parameter k must be determined to have a fully defined finger BP waveform beat. This parameter may be determined in various ways.

The parameter k may be determined via an empirical equation relating arterial area to fingertip dimensions (e.g., as measured with a smartphone as shown in FIG. 3B) or by calibration with a single cuff BP measurement. In either case, the arterial area may be assumed to be constant for a person.

Another way to determine the parameters is to detect diastolic BP (P_{d}) from the AC pressure waveform, similar to oscillometric algorithms like the fixed-ratio algorithm. Then, the minimum of ΔP(t) is scaled to equal P_{d}-Pₘ. Adding Pₘ to the waveform gives the finger BP waveform, and the peak of this waveform denotes systolic BP (Pₛ). Systolic BP is typically most difficult to measure via oscillometric algorithms.

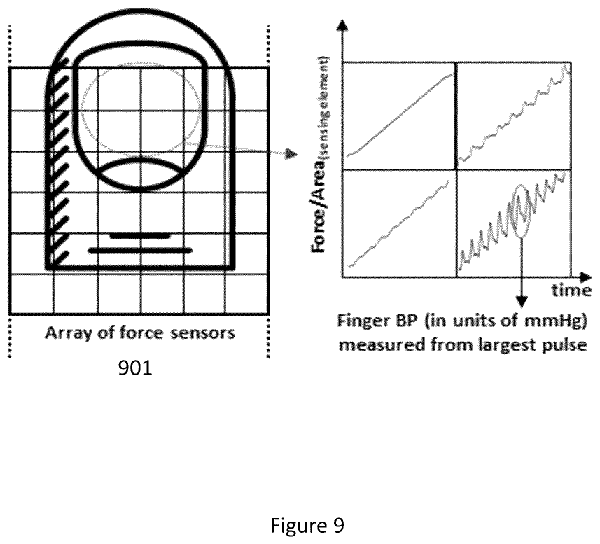

FIG. 9 shows a more accurate way to determine finger BP via tonometric finger pressing. A multi-sensor force array is attached to the back of a smartphone. The finger pressing actuation is performed. The maximum force oscillation beat over all finger pressures and all sensors is detected. This beat is divided by the sensing element area to yield a finger BP waveform beat. This method is algorithm-free, and it is the algorithm that limits the accuracy of oscillometric BP measurement. Additionally, since the sensing elements are smaller than the entire sensor, the pressure pulse for measurement is larger (and corresponds to BP via the applanation tonometry principle). As a result, each element does not need to be highly sensitive and could even have a resolution of around 1-2 mmHg with a range of 0-250 mmHg for example. If there is only one sensing element covering the same area, the maximum amplitude oscillation may only be a few mmHg, such that the resolution would have to be about 0.1-0.2 mmHg with the same range. Each sensing element can cover an area of 0.5 square mm or less. This example method could also be implemented with a mechanical contraption that automatically squeezes the fingertip.

In all cases, the finger BP waveform may be transformed to brachial BP and corrected to heart level as described in earlier sections.

Accurate BP Computation Algorithms via PPG and Sensitive Pressure Sensors

A sensitive pressure sensor for measuring the AC pressure waveform and DC external pressure may alternatively be used in conjunction with a PPG sensor to improve the accuracy of BP computation. As previously mentioned, oscillometric algorithms are a bottleneck in achieving clinical accuracy.

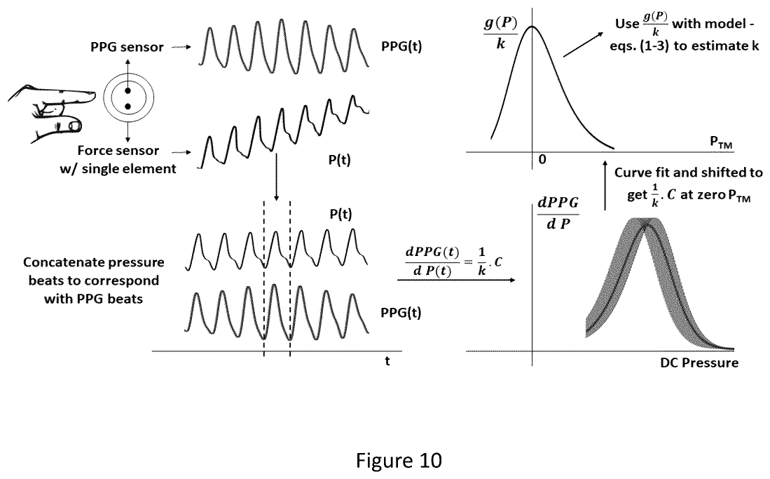

FIG. 10 shows PPG (AC component only) and sensitive pressure measurements during finger pressing actuation. The disclosed subject matter provides techniques to compute systolic and diastolic BP from the AC components of both PPG and pressure measurements.

Similar to an example method described in the preceding section, one way is to compute diastolic and mean BP from the oscillogram using the fixed-ratio or another algorithm, find the AC pressure beat of maximal amplitude, and scale it so that its minimum and mean equal diastolic and mean BP. Systolic BP is then given as the peak of the calibrated finger BP waveform.

Another way is to invoke a mathematical model of oscillometry. A useful oscillogram model represents the oscillation amplitude (ΔO) as the difference in the arterial blood volume-transmural pressure relationship (f(·)) evaluated at SP and DP (Pₛ and P_{d}) as follows:

ΔO=cf(Pₛ−Pₑ)−cf(P_{d}−Pₑ), (1)

where Pₑ is the external pressure of the artery and c is a scale factor to convert blood volume to the PPG units. Differentiating this equation with respect to P e yields the following model of the derivative of the oscillogram:

$$\frac{d Δ O}{{dP}_{e}} = c g ( {P}_{d} - {P}_{e} ) - c g ( {P}_{s} - {P}_{e} ) , \qquad ( 2 )$$

where g(·) is the derivative of f(·) and represents the arterial compliance curve. One exemplary parametric model for the compliance curve is as follows:

$$g ( P ) = γ {e}^{\frac{P}{α}} ( - \frac{P}{α} + 1 ) u ( - P ) + γ {e}^{- \frac{P}{β}} ( \frac{P}{β} + 1 ) u ( P ) , \qquad ( 3 )$$

where u(·) is the unit-step function, α and β reflect the arterial compliance curve widths over negative and positive transmural pressures, and γ denotes the height of the curve. This model may be fitted to a measured oscillogram alone to determine systolic and diastolic BP and the arterial compliance parameters. Hence, in contrast to conventional oscillometric algorithms, this example algorithm is specific to the person in the sense that both BP and compliance are measured. However, estimating five parameters from the limited information in the oscillogram can be challenging.

FIG. 10 also shows improved techniques to use the model in conjunction with the AC components of the PPG and pressure measurements to compute the BP levels. A PPG-force sensor unit, similar to that in FIG. 2A, is employed. However, this force sensor includes only one sensing element that is sensitive enough to detect the AC pressure waveform. The user performs the finger pressing method with the sensor unit. The AC pressure waveform beat of maximal amplitude is selected to get kΔP(t). This beat is concatenated to correspond with the multiple PPG waveform beats. To facilitate beat detection and alignment, an ECG waveform measured with dry electrodes on the same device can be leveraged. The derivative of the PPG waveform is then taken with respect to the concatenated pressure waveform and plotted against the DC pressure measurement to yield data points indicating a shifted arterial compliance curve, divided by k. A parametric function is fitted to the datapoints and the function is shifted, so that its peak is at zero transmural pressure (see Eq. (3)), to arrive at the scaled arterial compliance curve (i.e., g(·)/k). Either Eq. (1) or (2), with this curve substituted therein, is fitted to the measured oscillogram or its derivative in an optimal sense to estimate the reduced number of parameters (Pₛ, P_{d}, and k·c). Alternatively, the cross-correlation function between the compliance curve and the derivative of the oscillogram (perhaps after some smoothing) is taken. Consistent with Eq. (3), the peak location of the cross-correlation function denotes diastolic BP, and the valley location denotes systolic BP.

Mobile Devices for Venous Blood Pressure Measurement

All of the aforementioned examples pertain to arterial blood pressure (BP) measurement. However, venous blood pressure (VP) is also important. VP may be used to predict the onset of symptoms due to congestion in patients with heart failure (bilateral failure, right heart failure, or left heart failure leading to high pulmonary afterload) and thereby avert costly hospitalizations. VP may also be used to manage pulmonary arterial hypertension patients. However, VP typically requires invasive procedures for its measurement. The disclosed subject matter provides techniques to translate the above concepts for non-invasive measurement of VP.

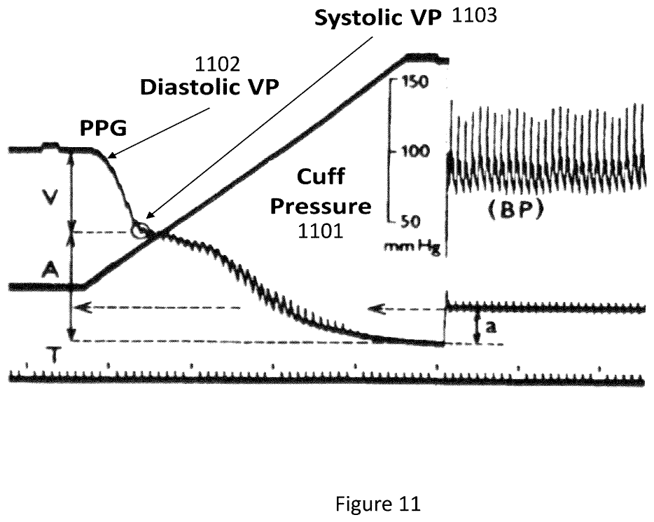

One method is based on FIG. 11, which shows the finger PPG waveform (AC+DC components) as a function of finger cuff pressure over the entire range of finger cuff pressures. (Here, the PPG waveform has been inverted in contrast to FIG. 5.) The plot shows an initial steep drop in PPG amplitude, followed by the common pattern of oscillometry. The two fiducial points on the steep drop may indicate diastolic and systolic VP. The idea is thus to vary the external pressure of the finger artery and measure the DC (and AC) component of the PPG waveform in addition to force to detect the VP. However, finger pressing may be difficult for the user to perform at very low contact pressures between 0 and 30 mmHg (corresponding to the VP range). Furthermore, at such low contact pressures, finger contact area should also be measured, as it will increase appreciably in this range (see FIG. 7).

To achieve these and other advantages, the disclosed subject matter provides techniques to create a finger worn ring sensor including an infrared PPG transducer for digital artery measurement, a force sensor with known contact area, and an accelerometer/gyroscope. It may be put on the finger with a Velcro strap or latching like a belt. The arm length of the person is measured to determine the pgh difference when the hand is at heart level versus fully lowered. The ring is tightened by the user to be roughly equal to this value under audio or visual guidance from the device. A marker may be used, such as belt hole number, to indicate the level of tightness for future use. Then, the user lowers their arm and slowly raises it to heart level as described earlier. The relative vertical height is measured with the accelerometer as also described previously. The plot of the DC and AC components of the PPG waveform versus the hydrostatic BP change minus the finger contact pressure of the sensor, which will range from 0 mmHg to pgh, is then used to detect VP.

Another solution for standard smartphones is to invoke the front camera and capacitive sensor array to measure the full PPG waveform, finger contact area, and finger force as described in previous sections. In this case, because VP is low, the useful ROI of the function relating area to force (see FIG. 7) may be interrogated to compute force from area. The user then uniformly presses their finger onto the camera and screen, starting at 0 mmHg and approaching 40-50 mmHg, to create a similar plot and detect VP. The finger pressing actuation may not be easy, but the device is readily available.

For both solutions, the AC component of the PPG may also be useful in detecting VP. For example, the maximal oscillation during the steep drop could be reflective of mean VP.

Another example method is based on the finger cuff volume clamping principle, also shown in FIG. 5. The principle is based on the arterial unloading concept. Arterial unloading is achieved when the external pressure of the vessel is set to the internal BP (i.e., zero transmural pressure). The blood volume in this unloaded state is not zero but rather a significant fraction (e.g., one-half) of the normal state, as arteries, unlike veins, are not collapsible vessels. An integrated cuff-PPG device is placed around the finger, and the measured blood volume is clamped to the unloaded state by quickly varying the cuff pressure through feedback (e.g., proportional-integral-derivative (PID) as shown in FIG. 5) control. As the blood volume rises/falls relative to the unloaded blood volume during systole/diastole, the cuff pressure increases/decreases almost instantly to keep the blood volume clamped at the unloaded blood volume (i.e., setpoint). This setpoint is initially determined in open-loop by slowly increasing the cuff pressure and invoking oscillometric algorithms. In this way, the cuff pressure may equal the finger BP waveform under closed-loop operation.

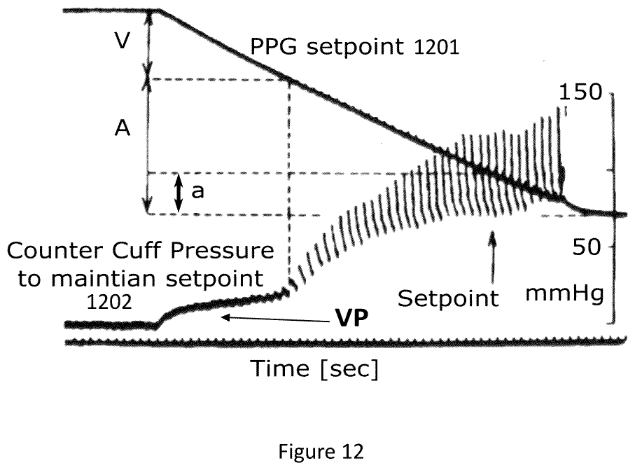

FIG. 12 shows a range of possible PPG setpoints and the corresponding finger cuff pressure required to maintain each setpoint. The cuff pressure trace slowly increases without much pulsatility and then begins to increase with greater pulsatility as the setpoint decreases. A lower setpoint is detected via oscillometric algorithms and used to measure BP. However, the cuff pressure at the higher setpoints indicates VP. Hence, the innovative concept is to create a finger cuff-PPG volume clamping device, vary the setpoint (e.g., over the higher range), and measure the cuff pressure required to maintain each setpoint. Then, the initial plateau region can be used as a measure of VP. The VP can be detected more accurately by identifying the cuff pressure that shows typical VP waveform character (e.g., a, c, x, v, and/or y waves). This method is more complicated but can be more accurate.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A device for determining blood pressure of a subject comprising:
a force sensor configured to measure finger pressure;
a camera configured to measure a finger photo-plethysmography (PPG) waveform;
a screen configured to display a visual indicator to guide a subject to place a side of a finger on the camera and the screen to target a digital artery and display the finger pressure in real time to guide the subject to uniformly press the finger on the camera and screen to vary external pressure of the artery; and
a processor configured to:
construct an oscillogram, wherein the oscillogram is a variable-amplitude blood volume oscillations versus external finger pressure function;
compute a blood pressure of the subject from the oscillogram; and
display the blood pressure on the screen.

2. The device of claim 1, wherein the processor is configured to determine the visual indicator based on different finger placements on the camera and the screen before performing the blood pressure measurement.

3. A device for determining blood pressure of a subject comprising:
a skin contact area sensor configured to measure a finger area;
a camera configured to measure a finger photo-plethysmography (PPG) waveform;
a screen configured to display a visual indicator to guide a subject to place a fingertip on the camera and the screen to target a transverse palmar arch artery and configured to display the finger pressure in real time to guide the subject to uniformly press the fingertip on the camera and screen to vary external pressure of the artery; and
a processor configured to:
convert the finger area to finger pressure based on a pre-defined nomogram;
construct an oscillogram, wherein the oscillogram is a variable-amplitude blood volume oscillations versus external finger pressure function;
compute systolic and diastolic blood pressures of the subject from the oscillogram; and
display the systolic and diastolic blood pressures on the screen.

4. The device of claim 3, wherein the nomogram is configured to determine finger force from the finger area based on selected parameters of a parametric function and divide the determined finger force by the finger area to determine finger pressure.

5. The device of claim 4, wherein the selected parameters are determined based on fingertip dimensions of the subject, a single cuff blood pressure reading, or a hand raising maneuver.

6. The device of claim 3, wherein the subject holds the device above heart level during the finger pressing for obtaining a more accurate nomogram, and wherein the processor is configured to use a vertical height between the device and a heart of the subject to adjust the blood pressure measurement to the heart level.

7. A device for determining blood pressure of a subject comprising:
a camera configured to measure a finger photo-plethysmography (PPG) waveform;
an accelerometer configured to measure a vertical height of the device relative to a heart of a subject;
an output device configured to guide the subject to raise a hand to vary the transmural pressure of an artery while maintaining a finger pressure on the camera; and
a processor configured to
compute pulse pressure of the subject from the finger PPG waveform and the vertical height; and
display the pulse pressure on the screen.

8. The device of claim 7, wherein the output device is further configured to
guide the subject to apply hard finger pressure on the camera;
guide the subject to change a level of finger pressure based on the measured AC and/or DC value of the PPG waveform and the PPG measurement during the hard finger pressure; and
the processor is further configured to identify a finger pressure corresponding to when a blood volume oscillation is near maximal.

9. The device of claim 8, wherein the processor is further configured to compare the PPG waveform during hand raising with the PPG waveform during finger pressing to assess a level of accuracy of the device.

10. The device of claim 7, wherein the processor is configured to construct a shifted oscillogram to relate variable-amplitude blood volume oscillations to a hydrostatic pressure change measured using the vertical height, wherein the pulse pressure is computed from the shifted oscillogram.

11. The device of claim 7, wherein the accelerometer is configured to measure the vertical height of the device relative to the heart.

12. The device of claim 7, wherein the processor is configured to convert the pulse pressure to brachial artery pulse pressure using a transfer function.

13. A device for determining blood pressure of a subject comprising:
a force sensor configured to measure finger pressure of the subject;
a finger photo-plethysmography (PPG) sensor configured to measure a finger PPG waveform of the subject;
a barometric pressure sensor configured to measure a vertical height of the device relative to a heart of the subject; and
a processor configured to:
measure readings of the barometric pressure sensor during finger pressing and without finger pressing while holding the device at heart level,
adjust the blood pressure measured during the finger pressing to the heart level using the readings of the barometric pressure sensor, and
display the adjusted blood pressure of the subject on a screen.

14. The device of claim 13, wherein the blood pressure is adjusted based on blood density, gravity, and/or the readings of the barometric sensor.

15. A device for determining blood pressure of a subject comprising:
a force sensor configured to measure finger pressure and finger pressure oscillation;
a visual indicator to guide a subject to place a fingertip on the force sensor;
a screen configured to display the finger pressure in real time to guide the subject to press a finger on the sensor to vary external pressure of an underlying artery; and
a processor configured to
measure AC and DC components of the finger pressure;
identify an AC finger pressure pulse of maximal oscillation and a DC finger pressure at the maximal oscillation;
determine a blood pressure of the subject based on the AC finger pressure pulse of maximal oscillation and the DC finger pressure at the maximal oscillation; and
display the blood pressure of the subject on the screen.

16. The device of claim 15, wherein the processor is further configured to determine the blood pressure based on fingertip dimensions of the subject and/or a single cuff blood pressure of the subject.

17. The device of claim 15, wherein the processor is configured to compute diastolic blood pressure from variable-amplitude finger pressure pulse oscillations and to compute systolic blood pressure from a blood pressure waveform.

18. The device of claim 17, wherein the blood pressure waveform is converted to the brachial artery blood pressure waveform using a transfer function and regression equation.

19. The device of claim 15, wherein the device further comprises a barometric pressure sensor to detect blood pressure at heart level.

20. A device for determining blood pressure of a subject comprising:
an array of force sensors configured to measure finger pressure and finger pressure pulse over each sensing element of the array;
a visual indicator to guide the person to place a fingertip of the subject on the sensor array;
a screen configured to display the finger pressure in real time to guide the subject to press the fingertip on the sensor to vary external pressure of an underlying artery; and
a processor configured to:
measure AC and DC components of the finger pressure at each sensing element of the array;
determine a blood pressure of the subject from the AC and DC components; and
display the blood pressure of the subject on the screen.

21. The device of claim 20, wherein the blood pressure is determined based on maximal pressure pulse oscillation over the sensing elements and the DC components of the finger pressures.

22. The device of claim 20, wherein the processor is further configured to generate a finger blood pressure waveform based on the AC and DC components and convert the blood pressure waveform to a brachial artery blood pressure waveform using a transfer function and regression model.

23. The device of claim 20, wherein the device further comprises a barometric pressure sensor to detect the blood pressure at heart level.

24. A device for determining blood pressure of a subject comprising:
a force sensor configured to measure finger pressure and a finger pressure pulse;
a finger photo-plethysmography (PPG) sensor configured to measure a PPG waveform;
a visual indicator to guide a subject in placing a fingertip on the force sensor and the PPG sensor;
a screen configured to display the finger pressure in real time to guide the subject to press the fingertip on the force sensor and the PPG sensor to vary the external pressure of the underlying artery; and
a processor configured to:
measure AC and DC components of the finger pressure and the PPG waveform;
compute an arterial compliance curve using an AC finger pressure component and PPG waveform;
compute a blood pressure of the subject using the arterial compliance curve; and
display the blood pressure of the subject on the screen.

25. The device of claim 24, wherein the processor is configured to:
compute the blood pressure by forming an oscillogram based on external finger pressure and the PPG waveform,
perform a cross-correlation between the arterial compliance curve and a derivative of the oscillogram with respect to pressure, and
determine a minimum value and a maximum value of the cross-correlation as systolic and diastolic blood pressures.
